(19) United States
(12) Patent Application Publication
(10) Pub. No.: US 2020/0146339 A1
(43) Pub. Date: May 14, 2020

(54) CANNABIS PRESS

(71) Applicant: Jacob Thomas Lanski, Ventura, CA (US)

(72) Inventor: Jacob Thomas Lanski, Ventura, CA (US)

(73) Assignee: Jacob Thomas Lanski, Ventura, CA (US)

(21) Appl. No.: 16/681,553

(22) Filed: Nov. 12, 2019

Related U.S. Application Data
(60) Provisional application No. 62/760,741, filed on Nov. 13, 2018.

(51) Int. Cl. A24D 1/14; A24B 3/14; A24D 1/18; A61K 36/185; A61J 3/00; B30B 11/04

(52) U.S. Cl. CPC A24D 1/14; A24B 3/14; B30B 11/04; A61K 36/185; A61J 3/00; A24D 1/18

(57) ABSTRACT

Provided herein are compressed plant material assemblies, as well as apparatuses and methods for forming compressed plant material assemblies. A compressed plant material assembly includes plant material such as smokable cannabis plant material. The plant material is compressed into a form such as a ball, with a wick disposed partially within the ball of compressed plant material. The apparatus includes a base, a lid, and a plunger. The base includes a mold cavity with a major portion for holding the plant material, and a minor portion for holding the wick partially inserted in the plant material. The lid, when applied to the base, does not enclose the major portion of the mold cavity, such that the plant material can be compressed with the plunger.

### CROSS-REFERENCES TO RELATED APPLICATIONS

The present application claims priority to U.S. Provisional Patent Application No. 62/760,741, filed Nov. 13, 2018, which application is incorporated herein by reference in its entirety.

### BACKGROUND

Presses and related devices are commonly used for compressing or crushing plant material such as herbs for culinary or medicinal purposes. These conventional devices most frequently are used to extract oils or juices from the compressed herbs, and are therefore not suitable for shaping or molding the herbs into a form from which the component oils have not been removed. In addition, existing plant presses are not generally designed for use with smokable herbs.

Typically, smokable plant material is sold in bulk and, prior to smoking the plant material, the user will portion off and condense the plant material, which can be time-consuming. Further, users typically burn the herbs with a lighter, which causes the herbs to reach undesirably high temperatures. For example, a butane lighter burns at around 3500° F. Burning plant material with a direct flame can destroy bonds in the plant material. For example, high temperatures can destroy cannabinoids and terpenes in cannabis, reducing the medical benefits of smoking the cannabis. The present disclosure solves this and other problems.

### BRIEF SUMMARY

In one aspect, the disclosure provides a compressed plant material assembly that includes a ball of compressed plant material; and a wick comprised of hemp, wherein a proximal end of the wick extends from the ball of compressed plant material and a distal end of the wick is disposed within the ball of compressed plant material. In certain aspects, the ball surrounds the wick.

In some embodiments, the compressed plant material assembly further includes concentrated plant material disposed within the ball of compressed plant material. In some embodiments, the ball of compressed plant material comprises cannabis. In some embodiments, the ball of compressed plant material has a diameter of approximately between about 3.9 millimeters (mm) and about 30.4 mm. In some embodiments, the compressed plant material assembly further comprises indicia marked on the ball of compressed plant material.

In some embodiments, the compressed plant material assembly is formed by a method comprising: providing the wick, plant material, a base, a lid, and a plunger, wherein the base has an upper surface comprising a mold cavity, wherein the mold cavity comprises a major portion having a substantially dome-like shape, wherein the mold cavity further comprises a minor portion having a substantially rectangular shape extending from the major portion along the upper surface, wherein the plunger comprises a proximal end and an opposite distal end, and wherein the distal end comprises a plunger cavity having a substantially dome-like shape configured such that the plunger cavity and the major portion of the mold cavity together form a substantially spherical cavity; laying the wick in the mold cavity, such that a portion of the wick is within the minor portion of the mold cavity, and such that a remainder of the wick is within the major portion of the mold cavity; placing the lid on top of the base, such that the lid does not enclose the major portion of the mold cavity; and compressing the plant material between the mold cavity and the plunger cavity using the plunger, thereby forming the ball of compressed plant material.

In some embodiments, the compressed plant material assembly is disposed, with one or more additional plant material assemblies, such that two or more compressed plant material assemblies are disposed within a container comprising two or more separate compartments configured to hold the two or more compressed plant material assemblies in the two or more respective separate compartments.

In another aspect, an apparatus for forming a compressed plant material assembly includes a base having an upper surface comprising a mold cavity, wherein the mold cavity comprises a major portion having a substantially dome-like shape, and wherein the mold cavity further comprises a minor portion having a substantially rectangular shape extending from the major portion along the upper surface; a lid configured for removable placement on top of the base, wherein upon such placement the lid does not enclose the major portion of the mold cavity; and a plunger comprising a proximal end and an opposite distal end, wherein the distal end comprises a plunger cavity having a substantially dome-like shape configured such that the plunger cavity and the major portion of the mold cavity together form a substantially spherical cavity.

In some embodiments, the base further comprises a shelf surrounding at least a portion of the mold cavity, and wherein the lid rests against the shelf upon placement of the lid on top of the base. In some embodiments, the major portion of the mold cavity further comprises indentations in a form of indicia to be marked on the compressed plant material assembly. In some embodiments, the apparatus further comprises a wick comprising hemp; and plant material comprising cannabis. In some embodiments, the base further comprises a chamber; and the mold cavity is included in a mold configured to removably sit at least partially inside the chamber of the base.

In some embodiments, the base further comprises one or more additional mold cavities disposed in an array of two or more mold cavities, each of the one or more additional mold cavities comprising a major portion having a substantially dome-like shape, and each of the one or more additional mold cavities further comprises a minor portion having a substantially rectangular shape extending from the major portion along the upper surface. In some embodiments, upon removable placement of the lid, the lid encloses the minor portion of each of the two or more mold cavities, of the array of two or more mold cavities, and wherein upon such placement the lid does not enclose the major portion of each of the two or more mold cavities. In some embodiments, the proximal end of the plunger comprises a scoop.

In another aspect, a method of forming a compressed plant material assembly includes providing a wick, plant material, a base, a lid, and a plunger, wherein the base has an upper surface comprising a mold cavity, wherein the mold cavity comprises a major portion having a substantially dome-like shape, and wherein the mold cavity further comprises a minor portion having a substantially rectangular shape extending from the major portion along the upper surface, wherein the plunger comprises a proximal end and an opposite distal end, and wherein the distal end comprises a plunger cavity having a substantially dome-like shape configured such that the plunger cavity and the major portion of the mold cavity together form a substantially spherical cavity; laying the wick in the mold cavity, such that a portion of the wick is within the minor portion of the mold cavity, and such that a remainder of the wick is within the major portion of the mold cavity, such that the lid does not enclose the major portion of the mold cavity; and compressing the plant material between the mold cavity and the plunger cavity using the plunger, thereby forming the compressed plant material assembly.

In some embodiments, the major portion of the mold cavity further comprises indentations in a form of indicia, and wherein the compressing further comprises marking the compressed plant material assembly with corresponding indicia. In some embodiments, the wick comprises hemp; and the plant material comprises cannabis.

In some embodiments, the method further comprises forming two or more compressed plant material assemblies by: providing one or more additional wicks such that two or more wicks are provided; providing one or more additional mold cavities such that two or more mold cavities are provided; laying each of the two or more wicks in one of the two or more mold cavities, such that a portion of the wick is within the minor portion of the mold cavity, and such that a remainder of the wick is within the major portion of the mold cavity; placing the lid on top of the base, such that the lid does not enclose the major portion of each of the two or more mold cavities; and compressing the plant material between the two or more mold cavities and the plunger cavity using the plunger, thereby forming the two or more compressed plant material assemblies. In some embodiments, the method further comprises disposing the two or more compressed plant material assemblies within a container comprising two or more separate compartments configured to hold the two or more compressed plant material assemblies in the two or more respective separate compartments.

### BRIEF DESCRIPTION OF THE DRAWINGS

FIGS. 1A and 1B illustrate a base of an apparatus for forming a compressed plant material assembly according to some embodiments.

FIGS. 2A and 2B illustrate a lid of an apparatus for forming a compressed plant material assembly according to some embodiments.

FIG. 3 illustrates a base of an apparatus for forming a compressed plant material assembly according to other embodiments.

FIG. 4 illustrates a mold of an apparatus for forming a compressed plant material assembly according to some embodiments.

FIG. 5 illustrates a lid of an apparatus for forming a compressed plant material assembly according to other embodiments.

FIGS. 6A-6C illustrate a plunger of an apparatus for forming a compressed plant material assembly according to some embodiments.

FIG. 7 illustrates an apparatus for forming a compressed plant material assembly according to some embodiments.

FIG. 8 illustrates a flowchart of a method in accordance with an embodiment for forming a compressed plant material assembly.

FIG. 9 illustrates a flowchart of a method in accordance with another embodiment for forming a compressed plant material assembly.

FIGS. 10A and 10B illustrate a compressed plant material assembly in accordance with some embodiments.

FIGS. 11A-11B illustrate a base of an apparatus for forming two or more compressed plant material assemblies, according to some embodiments.

FIGS. 12A-12B illustrate a lid of an apparatus for forming two or more compressed plant material assemblies, according to some embodiments.

FIG. 13 illustrates a base, lid, and plunger of an apparatus for forming two or more compressed plant material assemblies, according to some embodiments.

FIG. 14 illustrates a base, lid, and plungers of an apparatus for forming two or more compressed plant material assemblies, according to other embodiments.

FIGS. 15A-15C show a base, lid, and plungers of an apparatus for forming two or more compressed plant material assemblies according to other embodiments.

FIG. 16 presents a flowchart of a method 1600 in accordance with some embodiments for forming two or more compressed plant material assemblies.

FIG. 17 illustrates a system 1700 for forming and packaging compressed plant material assemblies, according to some embodiments.

### DETAILED DESCRIPTION

The present disclosure generally relates to a compressed plant material assembly, and a method and apparatus for compressing and shaping plant material into a compressed plant material assembly. The provided techniques are particularly useful for forming balls of smokable plant material such as, for example, cannabis. In some embodiments, the apparatus is for producing a single compressed plant material assembly, and provides advantageous properties that can include small size, high portability, and low manufacturing cost. In some embodiments, the apparatus is for providing multiple compressed plant material assemblies, and facilitate mass production thereof. Another benefit of the techniques disclosed herein is that the techniques enable the quick and easy generation of balls of plant material that each include a wick, allowing the balls to be more easily and readily used in smoking applications. In some aspects, the apparatuses include interchangeable molds that can be replaced as desired by the user to grant flexibility in the size or shape of compressed plant material that can be formed. These molds can also include patterns of indentations and/or protrusions that have been particularly configured to produce a printed effect on the surface of the compressed balls being formed. This feature can deliver the further advantage of marking produced balls with recognizable text, numerals, or images to deliver identifying or advertising information.

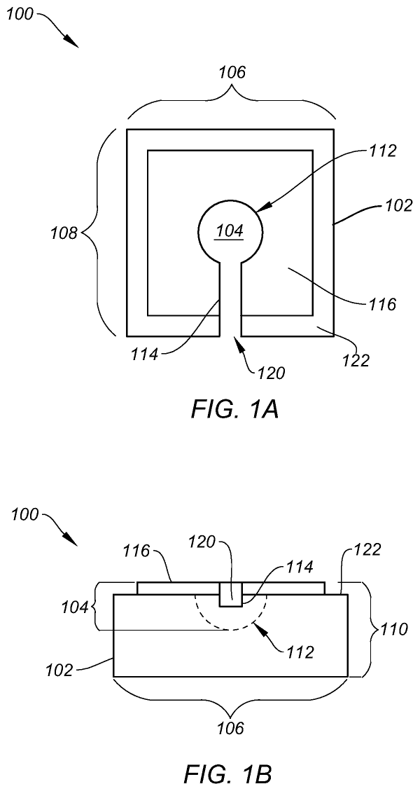

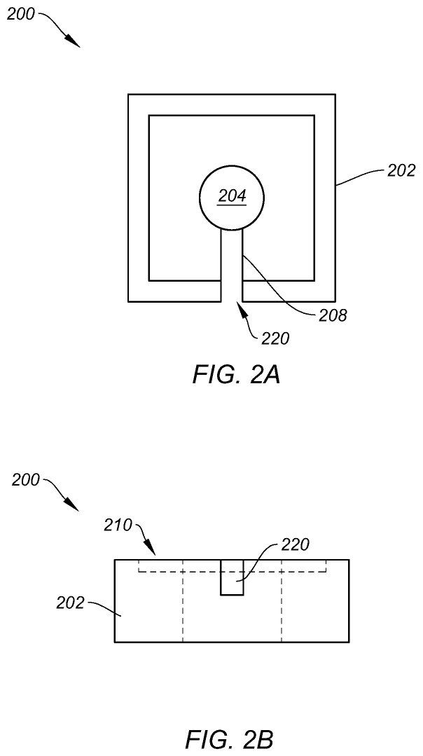

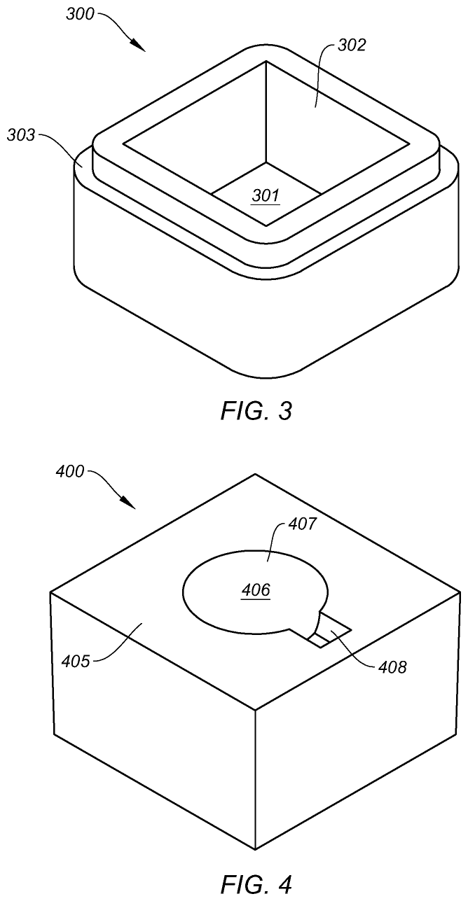

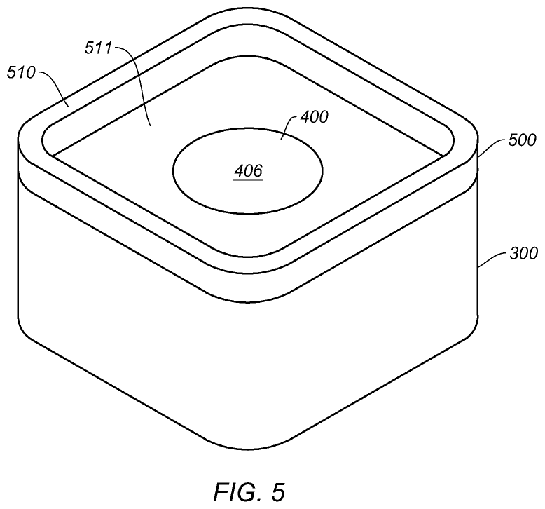

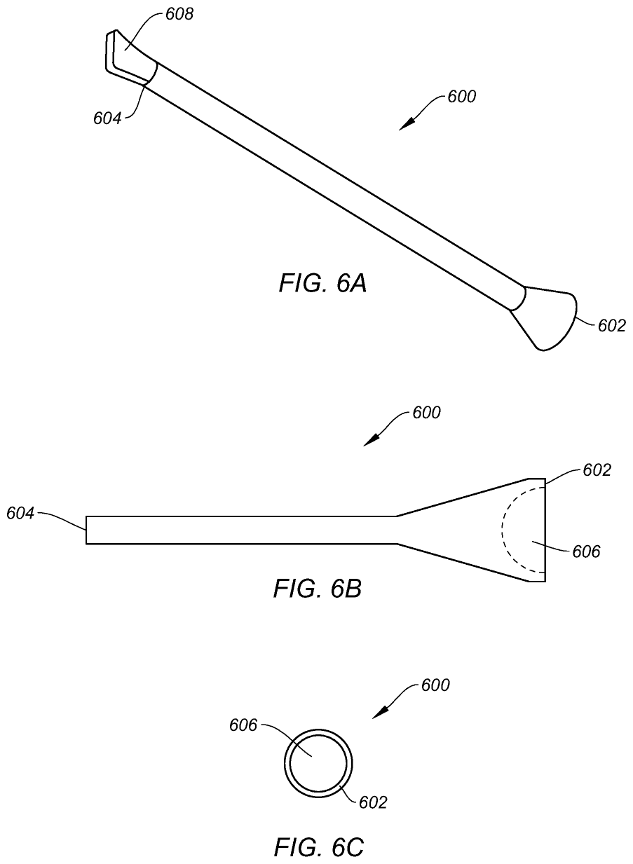

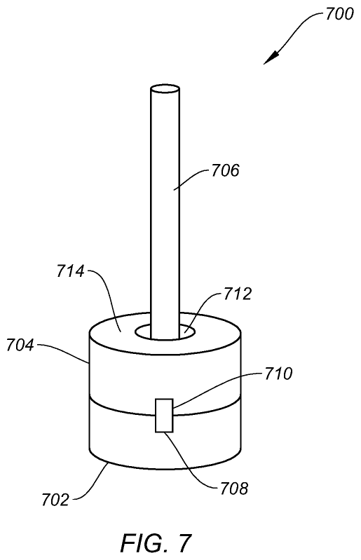

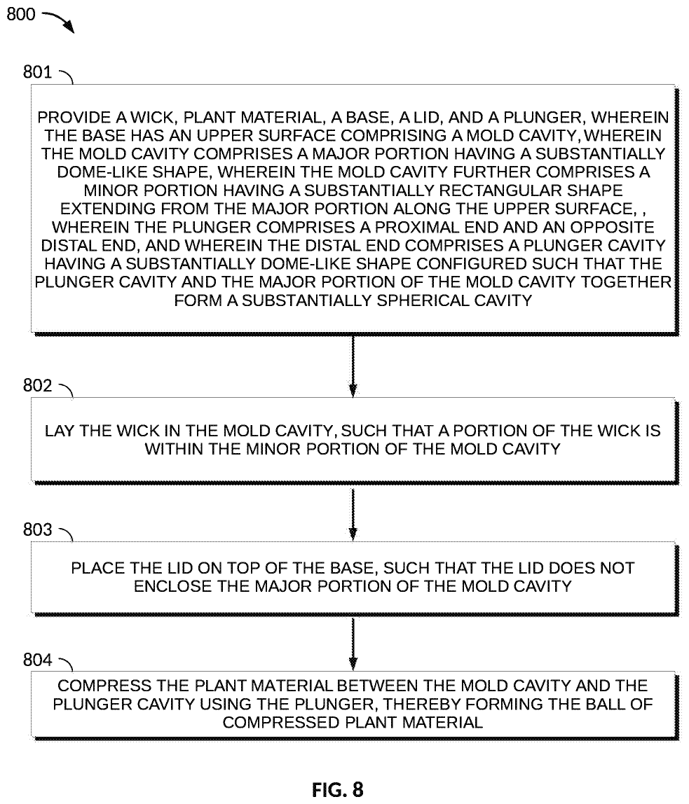

FIGS. 1A-7 illustrate individual and combined elements of an apparatus (which may also be referred to as a kit) for forming a compressed plant material assembly, according to some embodiments. FIGS. 1A and 1B show a base of the apparatus according to a first embodiment. FIGS. 2A and 2B show a lid component of the apparatus according to the first embodiment. FIGS. 3 and 4 show a separable base and mold, according to a second embodiment. FIG. 5 shows the base and mold of FIGS. 3 and 4, with a lid disposed thereon. FIGS. 6A-6C show a plunger component of the apparatus, according to some embodiments. FIG. 7 shows an apparatus including a base, lid, and plunger, according to some embodiments.

FIG. 1A illustrates a top view of a base 100 of the apparatus. FIG. 1B illustrates a side view of the base 100. The base 100 includes a housing 102 and a mold cavity 104. In some embodiments, and as shown in FIGS. 1A and 1B, the base 100 has a substantially rectangular footprint. Alternatively, the footprint of the base 100 can have a footprint with the shape of a circle, a triangle, another polygon, or an irregular form. For example, as shown in FIG. 7, the base may be cylindrical, with a circular footprint. The base 100 can be made of materials that include, for example, plastic, wood, metal (e.g., steel such as anodized steel), glass, or any combination thereof.

In some embodiments, the dimensions of the base 100 are selected to be small enough to provide a high degree of portability and a light weight, while being large enough to accommodate a suitably sized mold cavity 104 and to allow for easy handling. The base 100 may have a length 106, width 108, height 110 (as shown in FIG. 1B), and/or diameter, e.g., a characteristic dimension, of approximately between 0.5 centimeters (cm) and 10 cm, e.g., between 0.5 cm and 3 cm, between 1 and 2 cm, between 0.7 cm and 4.1 cm, between 0.9 cm and 5.5 cm, between 1.2 cm and 7.4 cm, or between 1.7 cm and 10 cm. In terms of upper limits, the base can have a characteristic dimension that is less than 10 cm, e.g., less than 7.4 cm, less than 5.5 cm, less than 4.1 cm, less than 3 cm, less than 2.2 cm, less than 1.7 cm, less than 1.2 cm, less than 0.9 cm, or less than 0.7 cm. In terms of lower limits, the base can have a characteristic dimension that is greater than 0.5 cm, e.g., greater than 0.7 cm, greater than 0.9 cm, greater than 1.2 cm, greater than 1.7 cm, greater than 2.2 cm, greater than 3 cm, greater than 4.1 cm, greater than 5.5 cm, or greater than 7.4 cm. Larger characteristic dimensions, e.g., greater than 10 cm, and smaller dimensions, e.g., less than 0.5 cm, are also contemplated.

In some embodiments, the base 100 includes a recessed shelf 122 extending along at least a portion of a perimeter of the base 100. In some embodiments, and as shown in FIG. 1A, the recessed shelf 122 extends along the entire perimeter of the base 100. In some embodiments, the recessed shelf 122 does not extend along the entire perimeter of the base 100.

In some embodiments, the mold cavity 104 is an indentation in the base 100 for shaping plant material. The mold cavity 104 may be manufactured as a permanent component of the base 100. In some embodiments, the mold cavity 104 includes a major portion 112 and a minor portion 114. Alternatively, the mold cavity may have a major portion 112, omitting the minor portion 114.

The major portion 112 of the mold cavity 104 is configured to have a shape and dimensions desired to be given to the formation of compressed plant material produced with the compressed plant material assembly. In some embodiments, the major portion of the mold cavity has a substantially dome-like (e.g., hemispherical) shape such that the compressed plant material formed with the kit will have a substantially ball-like (e.g., spherical) shape. Alternatively, the mold cavity may be in other shapes, such as a cube, prism, or pyramid.

The major portion 112 of the mold cavity 104 can have a diameter at the upper surface 116 of the mold cavity 104 that is, for example, between 2 millimeters (mm) and 60 mm, e.g., between 2 mm and 15.4 mm, between 8 mm and 12 mm, between 9.3 mm and 10.3 mm, between 2.8 mm and 21.6 mm, between 3.9 mm and 30.4 mm, between 5.5 mm and 42.7 mm, or between 7.8 mm and 60 mm. In terms of upper limits, the major portion opening diameter can be less than 60 mm, e.g., less than 42.7 mm, less than 30.4 mm, less than 21.6 mm, less than 15.4 mm, less than 11 mm, less than 7.8 mm, less than 5.5 mm, less than 3.9 mm, or less than 2.8 mm. In terms of lower limits, the major portion opening diameter can be greater than 2 mm, e.g., greater than 2.8 mm, greater than 3.9 mm, greater than 5.5 mm, greater than 7.8 mm, greater than 11 mm, greater than 15.4 mm, greater than 21.6 mm, greater than 30.4 mm, or greater than 42.7 mm. Larger diameters, e.g., greater than 60 mm, and smaller diameters, e.g., less than 2 mm, are also contemplated.

The minor portion 114 of the mold cavity 104 extends from the major portion 112 along the upper surface 116 of the mold cavity 104. In some embodiments, and as shown in FIGS. 1A-1B, the minor portion 114 has a substantially rectangular shape (e.g., the shape of a rectangular prism). The shape and dimensions of the minor portion 114 can be selected to accommodate a wick to be connected to and partially within the compressed plant material formed with the kit. In some embodiments, the minor portion 114 extends to the edge of the base 100, so as to form a side opening 120. Alternatively, the minor portion 114 may terminate within the base (e.g., in a similar fashion as illustrated in FIG. 4). In the finished product, the wick can be lit to ignite and combust or burn the compressed plant ball.

The minor portion 114 of the mold cavity 104 can have a length extending from the major portion 112 of the mold cavity 104 that is, for example, between 1 mm and 30 mm, e.g., between 1 mm and 7.7 mm, between 1.4 mm and 10.8 mm, between 2 mm and 15.2 mm, between 2.8 mm and 21.4 mm, or between 3.9 mm and 30 mm. In terms of upper limits, the minor portion 114 length can be less than 30 mm, e.g., less than 21.4 mm, less than 15.2 mm, less than 10.8 mm, less than 7.7 mm, less than 5.5 mm, less than 3.9 mm, less than 2.8 mm, less than 2 mm, or less than 1.4 mm. In terms of lower limits, the minor portion 114 length can be greater than 1 mm, e.g., greater than 1.4 mm, greater than 2 mm, greater than 2.8 mm, greater than 3.9 mm, greater than 5.5 mm, greater than 7.7 mm, greater than 10.8 mm, greater than 15.2 mm, or greater than 21.4 mm. Larger lengths, e.g., greater than 30 mm, and smaller lengths, e.g., less than 1 mm, are also contemplated.

The ratio of the major portion 112 diameter to the minor portion 114 length can be, for example, between 1 and 60, e.g., between 1 and 12, between 1.5 and 18, between 2.3 and 26, between 3.4 and 40, or between 5.1 and 60. In terms of upper limits, the ratio of the major portion diameter to the minor portion length can be less than 60, e.g., less than 40, less than 26, less than 18, less than 12, less than 7.7, less than 5.1, less than 3.4, less than 2.3, or less than 1.5. In terms of lower limits, the ratio of the major portion diameter to the minor portion length can be greater than 1, e.g., greater than 1.5, greater than 2.3, greater than 3.4, greater than 5.1, greater than 7.7, greater than 12, greater than 18, greater than 26, or greater than 40. Larger ratios, e.g., greater than 60, and smaller ratios, e.g., less than 1, are also contemplated.

In some embodiments, the major portion 112 of the mold cavity 104 includes indentations in the form of indicia. In some embodiments, the major portion 112 of the mold cavity 104 includes protrusions in the form of indicia. In some embodiments, the major portion 112 of the mold cavity 104 includes indentations and protrusions in the form of indicia. The indicia can be configured in the mold cavity 104 such that, when plant material is compressed using the mold cavity 104, complementary markings are produced on the surface of the formed ball.

The indicia can have the shape of one or more letters, numbers, patterns, images, or combinations thereof. In certain aspects, the indicia includes letters and/or numbers that can communicate information related to the plant material being formed into a ball with the apparatus. The plant material information can include, for example, a name of the plant material, a grade of the plant material, or an identification of the plant material supplier. In certain aspects, the indicia includes letters and/or numbers that can communicate information related to the ball formed with the apparatus. The ball information can include, for example, an approximate mass of the formed ball or an approximate volume of the formed ball. In certain aspects, the indicia includes letters and/or numbers that can communicate information related to the user of the kit. The user information can include, for example, an owner name or a dispensary name. In certain aspects, the indicia includes letters, numbers, and/or images that can advertise or promote an organization such as a school, a company, or a community. In some embodiments, the indicia includes a logo of an organization.

FIG. 2A illustrates a top view of a lid 200 of the apparatus. FIG. 2B illustrates a side view of the lid 200. The lid 200 may include an outer wall 202 and an upper opening 204. In some embodiments, the lid 200 may further include a mold cavity minor portion 208. Alternatively, the lid may not include a mold cavity minor portion 208. The lid 200 may further include a side opening 220. The mold cavity minor portion 208 of the lid 200 may terminate in the side opening 220. The side opening 220 of the lid 200 may be configured to align with the side opening 120 of the base, so as to form a combined opening through which a wick can be inserted, removed, and/or adjusted.

The lid 200 may be shaped, sized, and positioned such that the lid 200 can be removably attached to the base 100 (e.g., as shown in FIG. 7). Accordingly, the shape and dimensions of the lid 200 may be substantially similar to the shape and dimensions of the base 100, as described above with respect to FIGS. 1A and 1B. In some embodiments, when the lid 200 is disposed on the base 100, plant material can be inserted into the upper opening 204. In some embodiments, the lid rests against the shelf 122 upon placement of the lid 200 on top of the base 100.

In some embodiments, the lid 200 is configured such that, when the lid 200 is properly seated on top of the base 100, the mold cavity minor portion 114 of the base 100, and any wick inserted therein, are covered by the mold cavity minor portion 208 of the lid 200, and the mold cavity major portion is exposed. In some embodiments, when the lid is placed on top of the base 100, the lid 200 rests against the base 100. The lid 200 can be made of materials that include, for example, plastic, wood, metal (e.g., steel such as anodized steel), glass, or any combination thereof.

In some embodiments, the lid 200 and the base 100 may be permanently affixed to one another, or manufactured as a single piece. Accordingly, the apparatus may in some embodiments include a single lid-and-base assembly. In this case, by way of the upper opening 204 in the lid portion 200, plant material may be inserted. By way of the side openings 220 and 120 in the lid portion 200 and base portion 100 respectively, the wick may be inserted without removal of the lid 200.

FIGS. 3-5 illustrate individual and combined elements of an assembly for forming a compressed plant material, according to other embodiments. In the embodiment illustrated in FIGS. 3-5, the base 300 and mold 400 are separate components, which may be suitable if the user wishes to use different molds.

FIG. 3 shows a base 300 that acts as a lower or bottom component of the apparatus. Similarly as described above with respect to FIGS. 1A and 1B, the base may have a substantially rectangular footprint, or a footprint of other shapes, and can be made of materials that include, for example, plastic, wood, metal (e.g., steel such as anodized steel), glass, or any combination thereof. The dimensions of the base 300 may be similar to those described above with respect to the base 100 of FIGS. 1A and 1B.

The base 300 includes a chamber 301 having an open chamber top 302. The sides and bottom of the chamber 301 may be closed. In some embodiments, and as shown in FIG. 3, the chamber has a substantially prismatic shape, e.g., a substantially rectangular prismatic, triangular prismatic, or cubic shape. The chamber 301 can have the shape of a pyramid, a cylinder, a cone, a hemisphere, or an irregular form. The shape and dimensions of the chamber 301 are generally selected to match those of the mold component described below.

In some embodiments, the base 300 includes a recessed shelf 303 surrounding at least a portion of the chamber 301. In some embodiments, and as shown in FIG. 3, the shelf 303 is recessed such that the one or more walls of the chamber have a height that is greater than that of the shelf, and that the shelf completely surrounds the perimeter of the chamber. In some embodiments, the shelf 303 does not completely surround the perimeter of the chamber 301. The shelf can have a depth that is, for example, between 1 mm and 30 mm, e.g., between 1 mm and 7.7 mm, between 1.4 mm and 10.8 mm, between 2 mm and 15.2 mm, between 2.8 mm and 21.4 mm, or between 3.9 mm and 30 mm. In terms of upper limits, the shelf depth can be less than 30 mm, e.g., less than 21.4 mm, less than 15.2 mm, less than 10.8 mm, less than 7.7 mm, less than 5.5 mm, less than 3.9 mm, less than 2.8 mm, less than 2 mm, or less than 1.4 mm. In terms of lower limits, the shelf depth can be greater than 1 mm, e.g., greater than 1.4 mm, greater than 2 mm, greater than 2.8 mm, greater than 3.9 mm, greater than 5.5 mm, greater than 7.7 mm, greater than 10.8 mm, greater than 15.2 mm, or greater than 21.4 mm. Larger depths, e.g., greater than 30 mm, and smaller depths, e.g., less than 1 mm, are also contemplated.

FIG. 4 illustrates the mold 400 of the assembly of FIGS. 3-5. The mold 400 is configured to removably sit inside the chamber of the base 300 of FIG. 3. In this way, the same base 300 can be repeatedly used to accommodate different molds 400 as desired by the user of the apparatus. This can allow the user to swap molds 400 as needed in order to, for example, produce balls of compressed plant material having different shapes, dimensions, or markings. The exterior shape and dimensions of the mold 400 are generally selected to match those of the chamber described above. In some embodiments, and as shown in FIG. 1B, the mold 400 has a substantially prismatic shape, e.g., a substantially rectangular prismatic, triangular prismatic, or cubic shape. The mold 400 can have the shape of a pyramid, a cylinder, a cone, a hemisphere, or an irregular form. The mold 400 can be made of materials that include, for example, plastic, wood, metal, glass, or any combination thereof.

The upper surface 405 of the mold 400 includes a mold cavity 406 having a major portion 407 and a minor portion 408. The mold cavity 406, major portion 407, and minor portion 408 may be substantially similar to the mold cavity 104, major portion 112, and minor portion 114 of FIGS. 1A and 1B, but be disposed in the removable mold 400 rather than formed in the base 300.

FIG. 5 illustrates the lid 500 of the assembly of FIGS. 3-5. The lid 500 is depicted in FIG. 5 as placed on top of the base 300 and mold 400. In some embodiments, and as shown in FIG. 5, the lid 500 includes a raised outer wall 510 and a recessed tray 511. This configuration can improve the ease of handling and collecting excess plant material added to the mold cavity major portion 407. The lid is configured such that when it is properly seated on top of the base and the mold, the mold cavity minor portion, and any wick inserted therein, are covered by the lid, and the mold cavity major portion is exposed. In some embodiments, when the lid 500 is placed on top of the base 300 and the mold 400, the lid 500 rests against the shelf of the base 300. The lid 500 can be made of materials that include, for example, plastic, wood, metal, glass, or any combination thereof.

FIGS. 6A-6C illustrates a plunger 600, which may be used with one or more components of the apparatus as shown in FIGS. 1A-5. FIG. 6A illustrates a perspective view of a plunger 600. FIG. 6B illustrates a side view of a plunger 600, and FIG. 6C shows a front view of a plunger 600.

The plunger 600 includes a distal end 602 and an opposite proximal end 604. The distal end 602 of the plunger 600 includes a plunger cavity 606 that can be pressed by the user (or by an automated system) against any plant material held within the mold cavity major portion, thereby forming the compressed plant material. The plunger cavity 606 may be configured to have a shape and dimensions desired to be given to the formation of compressed plant material produced with the apparatus. Although other shapes are considered, in some embodiments, the plunger cavity 606 has a substantially dome-like (e.g., hemispherical) shape such that the compressed plant material formed with the apparatus will have a substantially ball-like (e.g., spherical) shape. In some embodiments, the plunger cavity 606 and the mold cavity major portion (e.g., 112 and 407 of FIGS. 1A, 1B, and 4, described above) substantially mirror one another, such that when the two are brought together, the plunger cavity 606 and the mold cavity major portion together form a substantially spherical (e.g., ball-like) cavity. In some embodiments the plunger cavity 606 may have a diameter (measured at the opening at the distal end) of between approximately 0.5 mm to approximately 50 mm (e.g., between approximately 1-10 mm, or between approximately 3-7 mm). The plunger cavity 606 may be sized and shaped in a substantially similar fashion to the mold cavity major portion, to generate a symmetrical ball of compressed plant material.

In some embodiments, the proximal end 604 of the plunger 600 includes a scoop or spatula 608, as illustrated in FIG. 6A. The inclusion of the scoop 608 on the plunger 600 can advantageously allow a user of the kit to more easily transfer plant material to the mold cavity major portion (e.g., 112 and 407) prior to compression of the plant material with the plunger cavity at the opposite distal end 602 of the plunger. Alternatively, or additionally, the plunger 600 may include measuring indicia (e.g., volume markers on the side of the scoop 608), to aid a user in portioning a desired amount of plant material. Alternatively, the proximal end 604 of the plunger 600 may not include a scoop, as shown in FIGS. 6B and 6C.

In some embodiments the plunger 600 can be stored within a slot in the base when not in use. The plunger can be made of materials that include, for example, plastic, wood, metal (e.g., steel such as anodized steel), glass, or any combination thereof.

In some embodiments, the plunger cavity 606 includes indentations and/or protrusions in the form of indicia. The indicia can be configured in the plunger cavity 606 such that, when plant material is compressed using the plunger cavity 606, complementary markings are produced on the surface of the formed ball. In some embodiments, the kit includes indentations and/or protrusions on both the plunger cavity 606 and the mold cavity major portion (e.g., 112 and 407). The plunger cavity 606 indicia can include any of the aforementioned types of letter, number, and/or image patterns, and can similarly be used for communication, identification, advertisement, decoration, or other purposes.

FIG. 7 illustrates an apparatus 700 which includes a base 702, a lid 704, and a plunger 706. The lid 704 may removably attach to the base 702, so that the attached components are flush with one another. Alternatively, the lid 704 and the base 702 may be a single component. In either event, the lid 704 and the base 702 may fit together, creating a smooth outer profile. Openings 708 and 710 of the base 702 and the lid 704, respectively, fit together, creating a combined opening in which a wick may be inserted. The plunger 706 fits in an opening 712 on a top surface 714 of the lid 704. In the embodiment pictured in FIG. 7, the base 702 and the lid 704 are substantially cylindrical in shape.

In some embodiments, the apparatus 700 may further include one or more sensors. The one or more sensors may detect an amount of mass in the apparatus 700. The sensors may generate an alert (e.g., the sensors may be coupled to a component which generates a beeping sound or flashing light, responsive to a signal from the sensor). Such an alert may be issued when a desired mass is detected (e.g., 1 gram, or any desired mass which in some cases may be configured by a user).

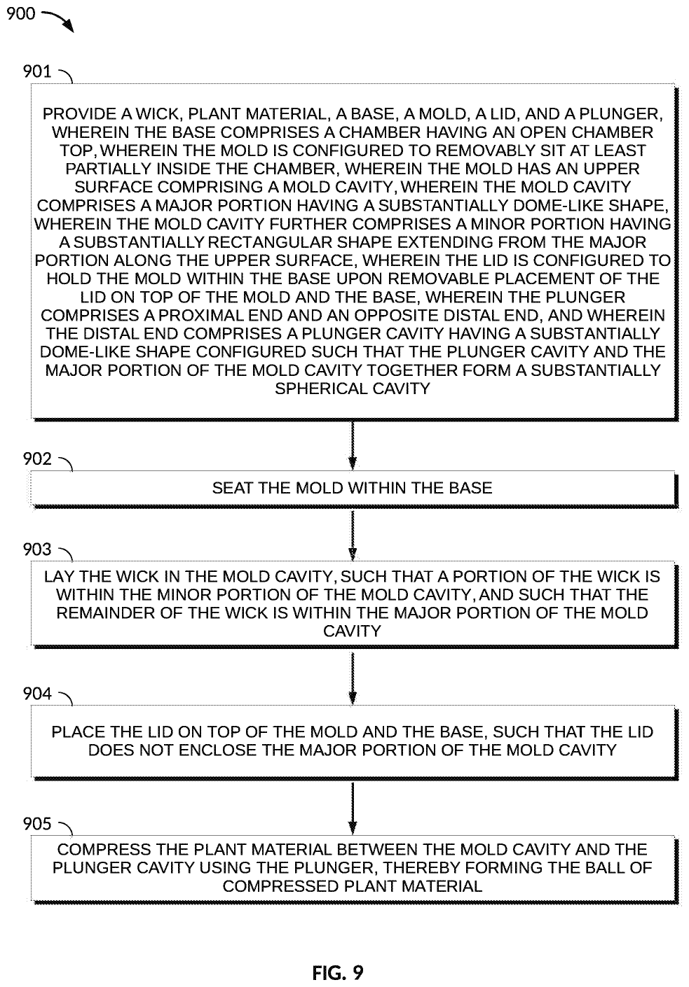

FIG. 8 is a flowchart of a method 800 in accordance with an embodiment for forming a compressed plant material assembly. The method 800 can be performed with an apparatus that includes a mold cavity integrated into the base as described above with respect to FIGS. 1A-2B, along with the plunger 600 of FIGS. 6A-6C.

In operation 801 of the method 800, a wick, plant material, a base, a lid, and a plunger are provided. The base has an upper surface that includes a mold cavity with a major portion having a substantially dome-like shape, and a minor portion having a substantially rectangular shape extending from the major portion along the upper surface. The lid may be configured for removable placement on top of the base. Alternatively, the lid may be permanently affixed to the base. The plunger includes a proximal end and an opposite distal end. The distal end includes a plunger cavity having a substantially dome-like shape configured such that the plunger cavity and the major portion of the mold cavity together form a substantially spherical cavity.

In operation 802 of the method 800, the wick is laid in the mold cavity, such that a portion of the wick is within the minor portion of the mold cavity, and the remainder of the wick, e.g., all of the wick not within the minor portion, is within the major portion of the cavity. Alternatively, the wick may extend beyond an opening in the side of the base and lid, such that a first portion of the wick is within the minor portion, a second portion of the wick is within the major portion, and a third portion of the wick extends out of the base and lid. In either event, some of the wick will be external to the formed ball of compressed plant material such that it is available for lighting, and some of the wick will be internal to the formed ball of compressed plant material. In some embodiments, laying the wick in the mold cavity includes disposing the wick the base prior to placing the lid thereupon. Alternatively, or additionally, laying the wick in the mold cavity may include sliding the wick into an opening (e.g., as shown in 708 and 710 of FIG. 7), either before or after placement of the lid.

In operation 803 of the method 800, the lid is placed on top of the base, such that the lid does not enclose the major portion of the mold cavity. The lid may partially, but not fully, cover the mold cavity. In this way, the major portion of the mold cavity is available for loading with plant material to be formed into the ball about a portion of the wick through compression. In some cases, the lid may enclose the minor portion of the mold cavity (e.g., to hold the wick in place). In some cases, the lid may not fully enclose the minor portion of the mold cavity (e.g. if there is a side opening for inserting the wick). In some embodiments (e.g., where the lid and base are permanently affixed to one another), this step may be omitted.

In operation 804 of the method 800, the plant material is compressed between the mold cavity and the plunger cavity using the plunger, thereby forming the ball of compressed plant material. For example, a user may press the plunger into the mold cavity. As another example, an automated system may press the plunger into the mold cavity (e.g., via an actuator coupled to a controller). In some embodiments, one or both of the mold cavity major portion and the plunger cavity includes indentations and/or protrusions for marking the surface of the formed ball as described above.

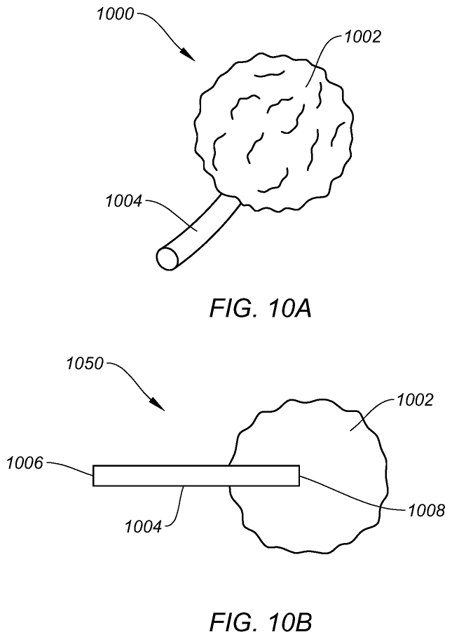

FIG. 9 is a flowchart of a method 900 in accordance with another embodiment for forming a compressed plant material assembly. The method can be used with, for example, the kit of FIGS. 3-6, wherein the kit includes a removable mold.

In operation 901 of the method 900, a wick, plant material, a base, a mold, a lid, and a plunger are provided. The base comprises a chamber having an open chamber top. The base can be the base of FIG. 3. The mold is configured to removably sit at least partially inside the chamber, and has an upper surface that includes a mold cavity with a major portion having a substantially dome-like shape, and a minor portion having a substantially rectangular shape extending from the major portion along the upper surface. The mold can be the mold of FIG. 4. The lid is configured to hold the mold within the base upon removable placement of the lid on top of the mold and the base. The lid can be the lid of FIG. 5. The plunger includes a proximal end and an opposite distal end. The distal end may include a plunger cavity having a substantially dome-like shape configured such that the plunger cavity and the major portion of the mold cavity together form a substantially spherical cavity. The plunger can be the plunger of FIGS. 6A-6C.

In operation 902 of the method 900, the mold is seated within the base. In some embodiments, the mold completely fits within the chamber of the base. In some embodiments, a portion of the mold fits within the chamber of the base.

In operation 903 of the method 900, the wick is laid in the mold cavity, such that a portion of the wick is within the minor portion of the mold cavity, and the remainder of the wick, e.g., all of the wick not within the minor portion, is within the major portion of the cavity. In this way, some of the wick will be external to the formed ball of compressed plant material such that it is available for lighting, and some of the wick will be internal to the formed ball of compressed plant material. As described above with respect to FIG. 8, the wick may be inserted into an opening or placed on top of the base and mold.

In operation 904 of the method 900, the lid is placed on top of the mold and the base, such that the lid does not enclose the major portion of the cavity. The lid may partially, but not fully, cover the mold cavity. In this way, and as exemplified by the illustration of FIG. 5, the major portion of the mold cavity is available for loading with plant material to be formed into the ball about a portion of the wick through compression. In some cases, the lid may fit on the base such that the lid encloses the minor portion of the mold cavity (e.g., to hold the wick in place). In some cases, the lid may not fully enclose the minor portion of the mold cavity (e.g. if there is a side opening for inserting the wick).

In operation 905 of the method 900, the plant material is compressed between the mold cavity and the plunger cavity using the plunger, thereby forming the compressed plant material assembly. In some embodiments, one or both of the mold cavity major portion and the plunger cavity includes indentations and/or protrusions for marking the surface of the formed ball as described above.

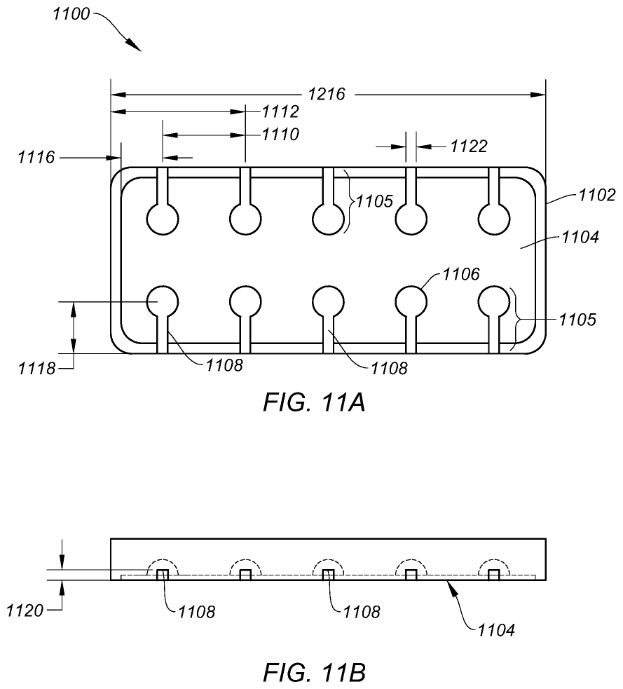

FIGS. 10A and 10B illustrate a compressed plant material assembly 1000, in accordance with some embodiments. FIG. 10A shows a perspective view of a compressed plant material assembly 1000. FIG. 10B shows a cross-section view of a compressed plant material assembly 1000. The compressed plant material assembly 1000 may be formed using the methods and apparatuses described herein.

FIG. 10A shows a compressed plant material assembly 1000 which includes a ball of compressed plant material 1002 and a wick 1004. The ball of compressed plant material 1002 may be substantially spherical in shape, as shown in FIG. 10A. Alternatively, the product of compressed plant material 1002 may have other shapes such as substantially elliptical, substantially cubic, and so forth. The ball of compressed plant material 1002 may include smokable plant material. The ball of compressed plant material 1002 may be composed of cannabis. Alternatively, or additionally, the ball of compressed plant material 1002 may be composed of other plant materials such as tobacco, mullein, moonwort, and so forth. In some embodiments, the compressed plant material assembly 1000 may further include concentrated plant material, such as cannabis concentrates, oils, and/or extracts (not pictured).

In some embodiments, the ball of compressed plant material 1002 may have a diameter of approximately between 2 millimeters mm and 60 mm, e.g., between 2 mm and 15.4 mm, between 8 mm and 12 mm, between 9.3 mm and 10.3 mm, between 2.8 mm and 21.6 mm, between 3.9 mm and 30.4 mm, between 5.5 mm and 42.7 mm, or between 7.8 mm and 60 mm.

The wick 1004 may include hemp. Alternatively, or additionally, the wick 1004 may include any flammable material, such as, for example, paper fibers, plant fibers, or combinations thereof. The wick 1004 can further include stiffeners or chemical treatments as desired and as generally known in the art. The wick may have a length of between approximately 1 mm and 100 mm, e.g., between 1.5 mm and 50 mm, between 1.3 mm and 15.2 mm, or between 3 mm and 26 mm.

FIG. 10B shows a cross-sectional view of a compressed plant material assembly 1000. As illustrated in FIG. 10B, the wick 1004 may partially or entirely extend into the ball of compressed plant material 1002. In FIG. 10B, the wick 1004 includes a distal end 1006 which extends from the ball of compressed material. The wick 1004 further includes a proximal end 1008 which is disposed within the ball of compressed plant material. The distal end 1006 of the wick 1004 may extend some distance (e.g., between approximately 1 mm and 30 mm) from the surface of the ball of compressed plant material 1002 to facilitate igniting the wick without contacting the compressed plant material with the ignition device (e.g., a lighter or match). The proximal end 1008 may extend some distance (e.g., between approximately 0.2 mm to 20 mm, as suitable based on the size of the ball of compressed plant material) into the ball of compressed plant material 1002 to facilitate burning the plant material, which may not require relighting the wick during the smoking of the compressed plant material.

The placement of the wick 1004 is advantageous because an ignition device such as a lighter or match can reach very high temperatures (e.g., around 3500 degrees Fahrenheit (° F.), which can be high enough to break down chemical bonds in some smokable plant materials. In contrast, a hemp wick burns at around 1500-1800° F. The higher the temperature, the greater the likelihood that smokable plant material may be combusted, which can destroy compounds such as terpenes and cannabanoids in cannabis. Thus, by burning the plant material at a lower temperature, additional medical benefits may be provided by preserving compounds in the plant material. As another advantage, the user need not apply a match or lighter to the smokable plant material directly or repeatedly. Direct and/or repeated application of a direct flame such as a match or lighter can create fumes (e.g., of wood or butane) which also may counteract medical benefits of smoking a particular plant material. Once lit, the wick may remain as a burning ember, obviating the need to repeatedly apply direct flame.

Further benefits are provided by the convenience of having single-serving compressed plant material assemblies. Commonly, if a user is going to smoke plant material, the user will manually pack down the plant material (e.g., with a finger, or some other implement such as a lighter). Packing the plant material to an appropriate density can ensure a smooth and regular flow of smoke. However, this can be time-consuming, and a user may find it difficult to manually achieve an appropriate density. The apparatus described herein can solve that problem by rapidly producing uniform compressed plant material assemblies. Further, the compressed plant material may be generated or purchased by the user ahead of time to streamline the process of preparing and smoking plant material. Accordingly, the compressed plant material assembly 1000 provides multiple advantages.

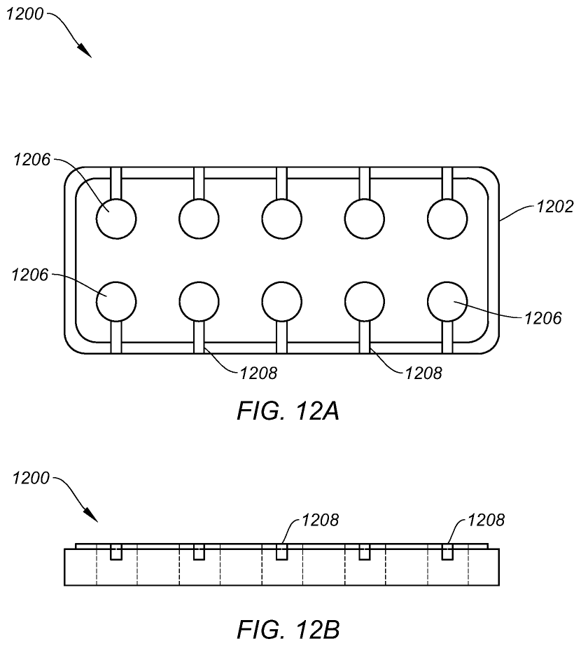

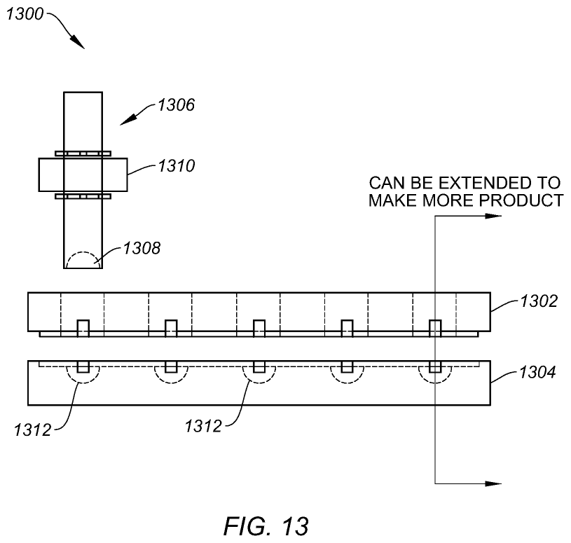

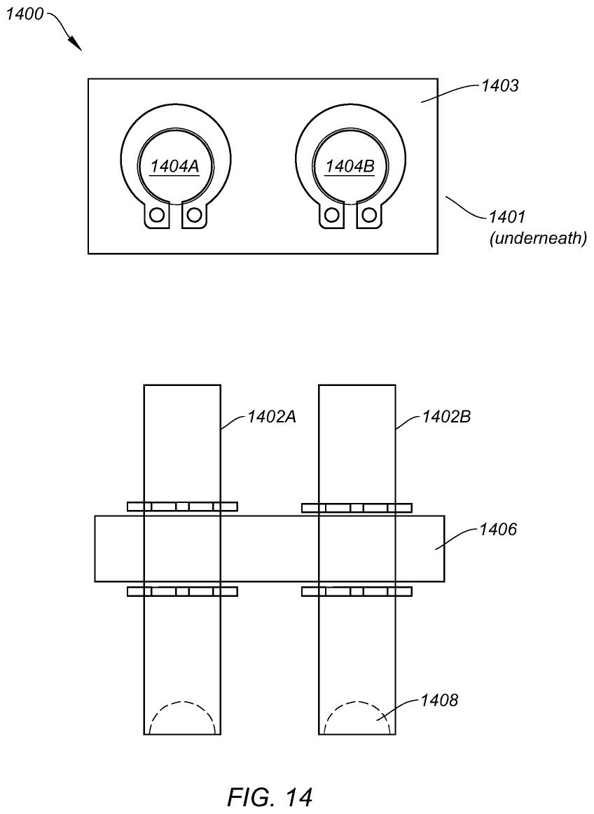

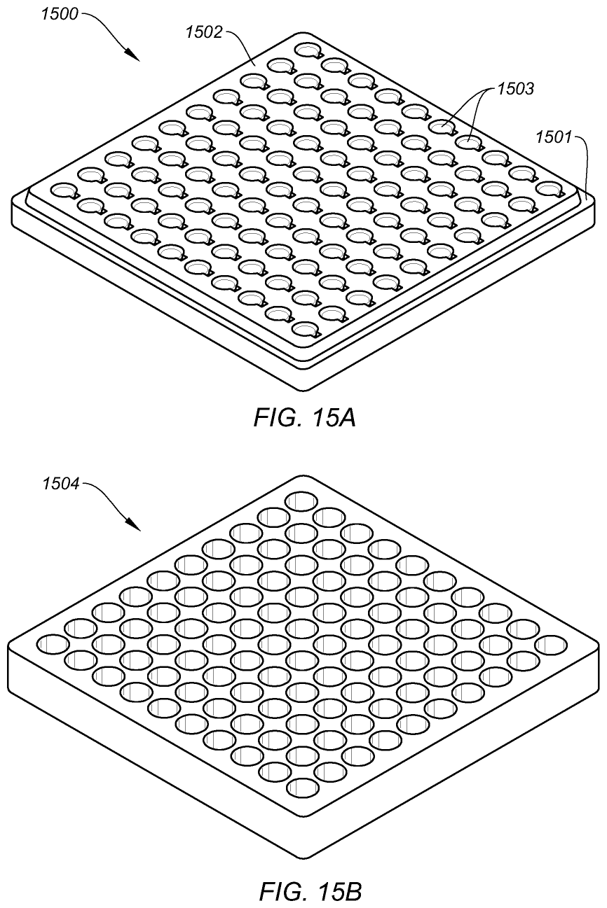

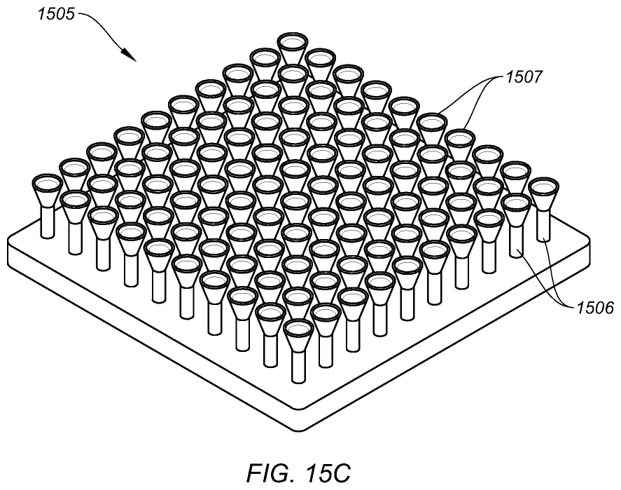

FIGS. 11A-15C illustrate individual and combined elements of an apparatus (which may also be referred to as a kit) for forming two or more compressed plant material assemblies, according to some embodiments. FIGS. 11A and 11B show a base of an apparatus for forming two or more compressed plant material assemblies according to some embodiments. FIGS. 12A and 12B show a lid of an apparatus for forming two or more compressed plant material assemblies according to some embodiments. FIG. 13 shows a base, lid, and plunger of an apparatus for forming two or more compressed plant material assemblies according to some embodiments. FIG. 14 shows a base, lid, and plungers of an apparatus for forming two or more compressed plant material assemblies according to other embodiments. FIGS. 15A-15C show a base, lid, and plungers of an apparatus for forming two or more compressed plant material assemblies according to other embodiments.

FIGS. 11A-12B illustrate individual elements of an apparatus for forming 10 balls of compressed plant material. FIGS. 11A and 11B illustrate views of a base of the apparatus, and FIGS. 12 A and 12B illustrate views of a lid of the apparatus.

FIG. 11A shows a top view of a base 1100, and FIG. 11B shows a side view of the base 1100. The base 1100 may act as a lower or bottom component of an apparatus for forming multiple (e.g., two or more) compressed plant material assemblies. In some embodiments, the base 1100 includes a recessed shelf 1102 extending along at least a portion of a perimeter of the base 1100. In some embodiments, and as shown in FIG. 11A, the recessed shelf 1102 extends along the entire perimeter of the base 1100. In some embodiments, the recessed shelf 1102 does not extend along the entire perimeter of the base 1100. In some embodiments, the width 1116 of the recessed shelf 1102 is in the range of approximately 1 mm to approximately 10 mm, e.g., approximately 2 mm to approximately 7 mm, or approximately 3 mm to approximately 5 mm.

In some embodiments, and as shown in FIG. 11A, the base 1100 has a substantially rectangular footprint. Alternatively, or additionally, the footprint of the base 1100 can have a footprint with the shape of a circle, a triangle, another polygon, or an irregular form. The base 1100 can be made of materials that include, for example, plastic, wood, metal (e.g., steel such as anodized steel), glass, or any combination thereof.

The upper surface 1104 of the base 1100 includes two or more mold cavities 1105, each having a major portion 1106 and a minor portion 1108 as described above with respect to FIGS. 1A and 1B. In some embodiments, the two or more mold cavities 1105 can each have shapes and dimensions that are identical to one another. Alternatively, the two or more mold cavities 1105 can include one or more mold cavities 1105 having a different shape and/or dimension than another one or more of the two or more mold cavities 1105. The two or more mold cavities 1105 can be positioned in a regular or irregular array in the upper surface of the base. For example, in the example illustrated in FIGS. 11A and 11B, the base 1100 may include 10 mold cavities 1105, with 5 mold cavities 1105 on each side of the base 1100.

The number of mold cavities 1105 in the base 1100 can be, for example, between 4 and 10,000, e.g., between 4 and 440, between 9 and 960, between 16 and 2100, between 25 and 4600, or between 100 and 10,000. In terms of upper limits, the number of mold cavities can be less than 10,000, e.g., less than 4600, less than 2100, less than 960, less than 440, less than 200, less than 100, less than 25, less than 16, or less than 9. In terms of lower limits, the number of mold cavities can be greater than 4, e.g., greater than 9, greater than 16, greater than 25, greater than 100, greater than 200, greater than 440, greater than 960, greater than 2100, or greater than 4600. Larger numbers of mold cavities, e.g., greater than 10,000, and smaller numbers of mold cavities, e.g., less than 4, are also contemplated.

In some embodiments, the mold cavities 1105 may be spaced apart from one another by a dimension 1110 of between approximately 0.1 cm and approximately 100 cm, e.g., between approximately 2 and approximately 3 cm, between approximately 0.5 and approximately 5 cm, or between approximately 1 cm and approximately 10 cm. In some embodiments, the mold cavities 1105 may be spaced apart from the outer edge of the base 1100 by a dimension 1112 of between approximately 0.1 cm and approximately 100 cm, e.g., between approximately 1 and approximately 2 cm, between approximately 0.5 and approximately 5 cm, or between approximately 1 cm and approximately 10 cm.

In some embodiments, the mold cavities 1105 may have a dimension 1118 of between approximately 0.1 cm and approximately 50 cm, e.g., between approximately 1 and approximately 2 cm, between approximately 0.5 cm and approximately 5 cm, or between approximately 1 cm and approximately 10 cm. In some embodiments, the minor portions 1108 of the mold cavities 1105 may have a height 1120 of between approximately 0.1 mm and approximately 20 mm, e.g., between approximately 4 mm and approximately 5 mm, between approximately 0.5 mm and approximately 5 mm, or between approximately 3 mm and approximately 8 mm. In some embodiments, the minor portions 1108 of the mold cavities 1105 may have a width 1122 of between approximately 0.1 mm and approximately 15 mm, e.g., between approximately 3 mm and approximately 4 mm, between approximately 0.5 mm and approximately 5 mm, or between approximately 2 mm and approximately 6 mm.

In some embodiments, the major portion 1106 of one or more of the two or more mold cavities 1105 include indentations and/or protrusions in the form of indicia as described above. In some embodiments, each of the two or more mold cavities 1105 includes indentations and/or protrusions in the form of indicia. The two or more mold cavities 1105 can each have indentations and/or protrusions that are identical to those of the other of the two or more mold cavities. The two or more mold cavities 1105 can include one or more mold cavities 1105 having different indentations and/or protrusions than those of another one or more of the two or more mold cavities 1105.

FIG. 12A shows a top view of a lid 1200, and FIG. 12B shows a side view of the lid 1200. The lid 1200 may act as a lower or bottom component of an apparatus for forming multiple (e.g., two or more) compressed plant material assemblies, and may be used with the base 1100 of FIGS. 11A and 11B.

In some embodiments, the lid 1200 includes an outer wall 1202, two or more upper openings 1206, and two or more side openings 1208. The lid 1200 may be configured such that when the lid 1200 is properly seated on top of the base 1100, each mold cavity minor portion 1108, and any wick inserted therein, are at least partially covered by the lid 1200, and each mold cavity major portion 1106 is at least partially exposed via the upper openings 1206. The side openings 1208 may facilitate insertion of the wicks and/or allowing the wicks to extend beyond the edge of the lid 1200.

In some embodiments, the lid 1200 is configured such that it aligns with the base 1100. Accordingly, the dimensions of the lid 1200 may be substantially equal to, and/or a mirror image of, the dimensions of the base 1100 described above. In some embodiments, when the lid is placed on top of the base, the lid rests against the shelf of the base. The lid can be made of materials that include, for example, plastic, wood, metal (e.g., steel such as anodized steel), glass, or any combination thereof.

FIG. 13 illustrates an apparatus 1300 for forming two or more compressed plant material assemblies, according to some embodiments. The apparatus 1300 includes a base 1304, a lid 1302, and a plunger 1306.

The base 1304 and the lid 1302 may be substantially similar to the base 1100 and lid 1200 described above with respect to FIGS. 11A-12B. As shown in FIG. 13, the base 1304 and the lid 1302 line up in operation to align for forming the compressed plant material assemblies. FIG. 13 illustrates an apparatus 1300 including five mold cavities 1312. As indicated in FIG. 13, the lid 1302 and the base 1304 may be extended to accommodate additional mold cavities 1312 for forming additional compressed plant material assemblies.

In some embodiments, the plunger 1306 may be actuated to insert into each of the two or more mold cavities of the base 1304. The plunger 1306 includes a plunger cavity 1308 that can be pressed against any plant material held within the major portion of the corresponding mold cavity, thereby forming the compressed plant material. The plunger cavity 1308 may have a shape and dimensions as described above. In some embodiments, the plunger cavity 1308 of the plunger 1306 may include indentations and/or protrusions in the form of indicia as described above.

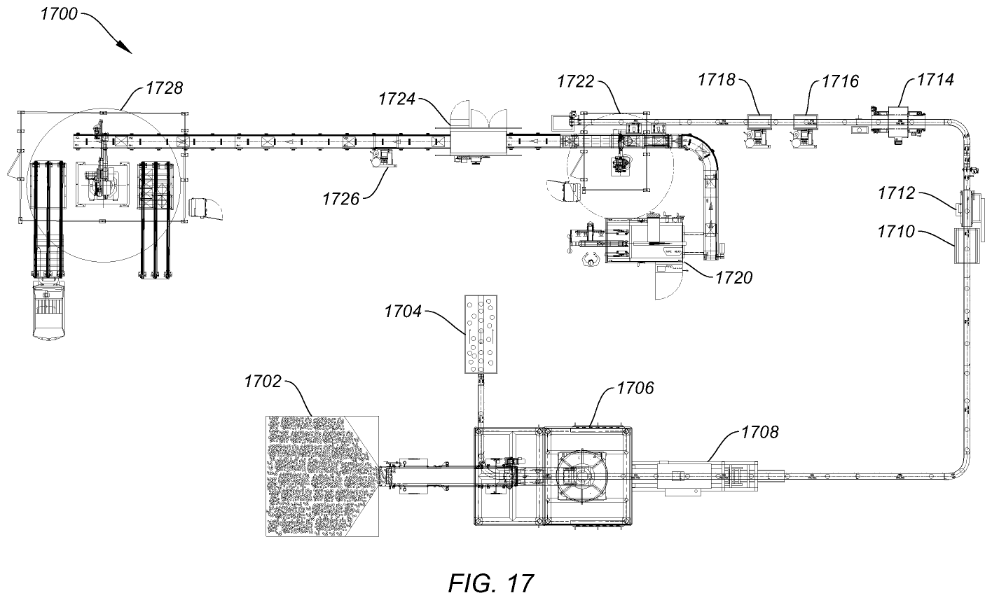

In some embodiments, the plunger 1306 includes an attachment means 1310 to attach the plunger 1306 to an automated actuator, such as an actuator arm coupled to a controller, as further described below with respect to FIG. 17. Accordingly, the plunger 1306 may be automatically actuated to compress plant material in each of the two or more mold cavities in an automated fashion. Accordingly, the apparatus 1300 can be used to quickly produce multiple compressed plant material assemblies in automated manufacture.

FIG. 14 illustrates another embodiment of an apparatus 1400 for forming two or more compressed plant material assemblies. The apparatus 1400 includes a base 1401 and lid 1403. The base 1401 includes two mold cavities 1404A and 1404B. The apparatus further includes two plungers 1402A and 1402B. Each plunger includes a plunger cavity 1408.

In the embodiment illustrated in FIG. 14, two plungers 1402A and 1402B are provided. Although two plungers 1402A and 1402B are shown for simplicity of illustration, it should be understood that additional mold cavities may be provided, such that two or more plungers are provided. One plunger may be provided for each mold cavity 1404. For example, there can be 12 mold cavities and 12 plungers in some embodiments, 50 mold cavities and 50 plungers in some embodiments, and so forth. The plungers 1402A and 1402B are coupled to a single attachment means 1406. Accordingly, an actuator arm can move the two or more plungers 1402A and 1402B in concert, such that multiple compressed plant material assemblies may be formed substantially simultaneously, speeding up the process even further.

FIGS. 15A-15C illustrate another example of a kit for forming two or more compressed plant material assemblies, according to some embodiments. FIGS. 15A-15C illustrate individual elements of a kit for forming 100 balls of compressed plant material. The image in FIG. 15A is of a base 1500 that acts as a lower or bottom component of the kit. In some embodiments, the base includes a recessed shelf 1501. The base 1500 and recessed shelf 1501 may be substantially similar to the base 1100 and recessed shelf 1102 described above with respect to FIG. 11A. However, in this example, the base 1500 includes 100 mold cavities 1503.

The upper surface 1502 of the base 1500 includes two or more mold cavities 1503, each having a major portion and a minor portion as described above. The two or more mold cavities 1503 can each have shapes and dimensions that are substantially identical to one another. The two or more mold cavities 1503 can include one or more mold cavities having a different shape and/or dimension than another one or more of the two or more mold cavities. The two or more mold cavities can be positioned in a regular or irregular array in the upper surface of the base.

The number of mold cavities in the base can be, for example, between 4 and 10,000, e.g., between 4 and 440, between 9 and 960, between 16 and 2100, between 25 and 4600, or between 100 and 10,000. In terms of upper limits, the number of mold cavities can be less than 10,000, e.g., less than 4600, less than 2100, less than 960, less than 440, less than 200, less than 100, less than 25, less than 16, or less than 9. In terms of lower limits, the number of mold cavities can be greater than 4, e.g., greater than 9, greater than 16, greater than 25, greater than 100, greater than 200, greater than 440, greater than 960, greater than 2100, or greater than 4600. Larger numbers of mold cavities, e.g., greater than 10,000, and smaller numbers of mold cavities, e.g., less than 4, are also contemplated. In some embodiments, and as shown in FIG. 15A, the base includes a regular array of 100 mold cavities.

In some embodiments, the major portion of one or more of the two or more mold cavities 1503 include indentations and/or protrusions in the form of indicia as described above. In some embodiments, each of the two or more mold cavities 1503 includes indentations and/or protrusions in the form of indicia. The two or more mold cavities 1503 can each have indentations and/or protrusions that are identical to those of the other of the two or more mold cavities. The two or more mold cavities 1503 can include one or more mold cavities having different indentations and/or protrusions than those of another one or more of the two or more mold cavities.

FIG. 15B illustrates the lid 1504 of the kit of FIGS. 15A-15C. The lid 1504 is depicted in FIG. 15B as placed on top of the base 1500. The lid 1504 is configured such that when it is properly seated on top of the base, each mold cavity minor portion, and any wick inserted therein, are covered by the lid, and each mold cavity major portion is exposed. In some embodiments, when the lid 1504 is placed on top of the base 1500, the lid 1504 rests against the shelf of the base 1500. The lid 1504 can be made of materials that include, for example, plastic, wood, metal (e.g., steel such as anodized steel), glass, or any combination thereof.

FIG. 15C illustrates a plunger board 1505 of the kit of FIGS. 15A-15C. The plunger board 1505 includes one plunger 1506 for each of the two or more mold cavities of the base 1500. The number and arrangement of the two or plungers 1506 are selected to mirror those of the two or more mold cavities 1503. Each of the two or more plungers 1506 includes a plunger cavity 1507 that can be pressed by a user, or automatically, against any plant material held within the major portion of the corresponding mold cavity, thereby forming the compressed plant material. Each of the two or more plunger cavities can have a shape and dimensions as described above.

In some embodiments, the plunger cavity of one or more of the two or more plungers include indentations and/or protrusions in the form of indicia as described above. In some embodiments, each of the two or more plunger cavities includes indentations and/or protrusions in the form of indicia. The two or more plunger cavities can each have indentations and/or protrusions that are identical to those of the other of the two or more mold cavities. The two or more plunger cavities can include one or more mold cavities having different indentations and/or protrusions than those of another one or more of the two or more plunger cavities. In some embodiments, the kit includes indentations and/or protrusions on one or more plunger cavities and one or more mold cavities.

In some embodiments, in a similar fashion as described above with respect to FIGS. 1A-2B, the base and the lid may be fused together in the case of an apparatus with multiple mold cavities. This may result in a single base-and-mold apparatus, with multiple openings for inserting plant material and/or wicks.

In some embodiments, the apparatus may have a different shape other than the substantially rectangular footprints shown. In one embodiment, a substantially cylindrical, integrated base and lid is provided, with the mold cavities disposed on an outer side of the cylindrical, integrated base and lid. The plunger board may further be substantially cylindrical in shape, with a plurality of plungers disposed on an outer side of the cylindrical plunger board. The cylindrical plunger board and the cylindrical integrated base and lid may be coupled to one or more motors, which causes rotation of the cylindrical plunger board and the cylindrical integrated base. The positions of the cylindrical plunger board and the cylindrical integrated base may be such that, when rotated, the plungers, in turn, press the respective mold cavities, generating balls of compressed plant material. Plant material may be disposed between the two rolling cylinders (e.g., dropped via a hopper as shown in FIG. 17), for rapid automatic generation of the compressed plant material assemblies.

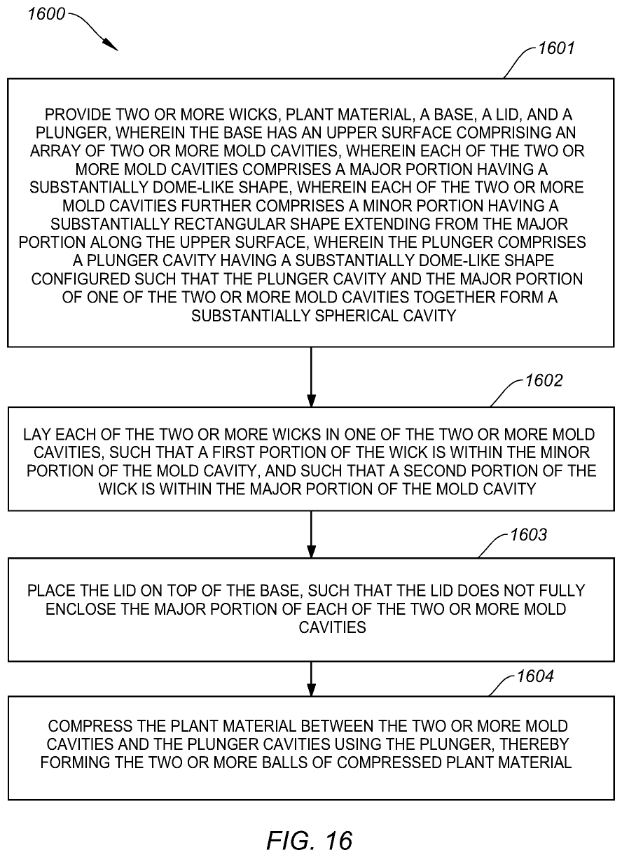

FIG. 16 presents a flowchart of a method 1600 in accordance with some embodiments for forming two or more compressed plant material assemblies. The method 1600 can be used with, for example, the kits as illustrated in any of FIGS. 11A-15C.

In operation 1601 of the method 1600, two or more wicks, plant material, a base, a lid, and a plunger are provided. The base has an upper surface that includes an array of two or mold cavities. Each mold cavity includes a major portion having a substantially dome-like shape, and a minor portion having a substantially rectangular shape extending from the major portion along the upper surface. The base can be, for example, the base of FIG. 11A or the base of FIG. 15A. In some embodiments, the lid is configured for removable placement on top of the base. Alternatively, the lid may be permanently affixed to the base. The lid can be, for example, the lid of FIG. 12A or the lid of FIG. 15B.

In some embodiments, a single plunger may be provided, as illustrated in FIG. 13. Alternatively, multiple plungers may be provided, as illustrated in FIGS. 14 and 15C. In some embodiments, as shown in FIG. 15C, the plungers may be provided on a plunger board, which includes an array of one plunger for each of the two or more mold cavities. In any case, each plunger includes a plunger cavity having a substantially dome-like shape configured such that the plunger cavity and the major portion of one of the two or more mold cavities together form a substantially spherical cavity.

In operation 1602 of the method, each of the two or more wicks is laid in one of the two or more mold cavities, such that a portion of the wick is within the minor portion of the mold cavity, and the remainder of the wick, e.g., all of the wick not within the minor portion, is within the major portion of the mold cavity. In this way, some of the wick will be external to the formed ball of compressed plant material such that it is available for lighting, and some of the wick will be internal to the formed ball of compressed plant material. Alternatively, in some cases, not all of the wick is within the minor portion. For example, the wick may extend through an opening and exit the base and lid. Laying the wicks in the mold cavities may include disposing the wicks on the mold cavities (e.g., by a user or a robotic system) prior to placing the lid. Alternatively, or additionally, laying the wicks in the mold cavities may include inserting the wicks in slots in the apparatus (e.g., by a user or a robotic system).

In operation 1603 of the method, the lid is placed on top of the base, such that the lid does not fully enclose the major portion of each of the two or more mold cavities. In this way, and as exemplified by the illustration of FIGS. 14 and 15B, the major portion of each mold cavity is available for loading with plant material to be formed into the ball about a portion of the wick through compression. As described above, openings in the lid may expose the major portion of each mold cavity in whole or in part. Further, the top and bottom of the minor portion of each mold cavity may be enclosed in whole or in part by the lid, depending on whether openings are present in the sides of the base and lid, as illustrated in FIGS. 11A-11B. Accordingly, in some embodiments, instead of laying the wick on the base prior to placing the lid thereupon, the wick may be inserted through such side openings.

In operation 1604 of the method, the plant material is compressed between the two or more mold cavities and the plunger cavities using the plunger, thereby forming the two or more balls of compressed plant material. For example, using the apparatus illustrated in FIG. 13, a single plunger may be actuated to compress the material in each of the two or more mold cavities in turn. As another example, using the apparatus illustrated in FIG. 14, two or more plungers may be jointly actuated to compress the material in two or more mold cavities substantially simultaneously. As another example, using the apparatus illustrated in FIGS. 15A-15C, a plunger board may be pressed on the lid and the base, compressing the plant material in two or more mold cavities substantially simultaneously. As yet another example, cylindrical components may be rotated to compress the plant material between the mold cavities and the plunger cavities.

In some embodiments, one or more of the two or more mold cavity major portions and/or one or more of the plunger cavities includes indentations and/or protrusions for marking the surface of one or more of the formed balls as described above.

In some embodiments, the method 1600 may further include disposing the two or more plant material assemblies within a container comprising two or more separate compartments configured to hold the two or more plant material assemblies in the two or more respective separate compartments. The formed plant material assemblies may be disposed into a container similar to an egg carton or pill dispenser, which keeps the plant material assemblies separated and protected.

FIG. 17 illustrates a system 1700 for forming and packaging compressed plant material assemblies, according to some embodiments. The system 1700 may be used to automatically generate compressed plant material assemblies, using the components and methods described above.

The system 1700 may include one or more of the following components including a raw product or feed stock infeed 1702, an empty container loading component 1704, a raw product scale 1706, a product forming and loading component 1708, a tamper evident applicator 1710, a top cap applicator 1712, a metal detector 1714, a retail label applicator 1716, a Julian code applicator 1718, a case erector 1720, a robotic case packing cell 1722, a case sealer 1724, a case labeler 1726, and a robotic palletizing cell 1728. The system 1700 may work counterclockwise from 1702 to 1728 to produce and package large quantities of compressed plant material assemblies. The system 1700 may further include one or more controllers and/or computing devices to provide instructions to operate the components thereof.

The raw product infeed 1702 may include a hopper and dispenser for holding bulk quantities of compressed plant material. For example, the raw product infeed 1702 may be filled with cannabis, and dispense cannabis for forming compressed plant material assemblies.

The empty container loading component 1704 may provide empty containers for storing the compressed plant material assemblies. The raw product scale 1706 may weigh raw product (e.g., cannabis or other plant material), so that uniform amounts of raw product may be used to form the compressed plant material assemblies.

The product forming and loading component 1708 may include functionality to form one or more compressed plant material assemblies, as described in detail above with respect to FIGS. 1A-16. The product forming and loading component 1708 may further include functionality to load the formed compressed plant material assemblies into the empty containers provided by the empty container loading component 1704.

The tamper evident applicator 1710 may apply tamper evident elements, and the top cap applicator 1712 may apply a top cap to the filled container. The metal detector 1714 may be used for identifying unwanted metals in the product.

The retail label applicator 1716 may apply retail labels to the containers, which may specify information such as the type of plant material, a brand name, a price, and so forth. The Julian code applicator 1718 may apply a Julian date to the containers.

The remaining components of the system 1700 may package the containers for transport, e.g., multiple containers to a case and multiple cases to a pallet. The case erector 1720 may place cases in an upright position for packing. The robotic case packing cell 1722 may fill the cases with containers of compressed plant material assemblies (e.g., via a robotic arm coupled to a controller). The case sealer 1724 may seal the cases, e.g., by robotically applying tape or other adhesive. The case labeler 1726 may apply labels to the cases. The robotic palletizing cell 1728 may load the packed cases onto pallets for transport. Accordingly, the system 1700 can be used to produce large amounts of compressed plant material assemblies quickly, efficiently, and automatically.

Although specific embodiments have been described, various modifications, alterations, alternative constructions, and equivalents are possible. Additionally, although certain embodiments have been described using a particular series of steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described embodiments may be used individually or jointly.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compressed plant material assembly comprising:
a ball of compressed plant material; and
a wick comprised of hemp, wherein a proximal end of the wick extends from the ball of compressed plant material and a distal end of the wick is disposed within the ball of compressed plant material.

2. The compressed plant material assembly of claim 1, further comprising concentrated plant material disposed within the ball of compressed plant material.

3. The compressed plant material assembly of claim 1, formed by a method comprising:
providing the wick, plant material, a base, a lid, and a plunger,
wherein the base has an upper surface comprising a mold cavity, wherein the mold cavity comprises a major portion having a substantially dome-like shape, wherein the mold cavity further comprises a minor portion having a substantially rectangular shape extending from the major portion along the upper surface,
wherein the plunger comprises a proximal end and an opposite distal end, and wherein the distal end comprises a plunger cavity having a substantially dome-like shape configured such that the plunger cavity and the major portion of the mold cavity together form a substantially spherical cavity;
laying the wick in the mold cavity, such that a portion of the wick is within the minor portion of the mold cavity, and such that a remainder of the wick is within the major portion of the mold cavity;
placing the lid on top of the base, such that the lid does not enclose the major portion of the mold cavity; and
compressing the plant material between the mold cavity and the plunger cavity using the plunger, thereby forming the ball of compressed plant material.

4. The compressed plant material assembly of claim 1, wherein the ball of compressed plant material comprises cannabis.

5. The compressed plant material assembly of claim 1, the ball of compressed plant material having a diameter of approximately between about 3.9 millimeters (mm) and about 30.4 mm.

6. The compressed plant material assembly of claim 1, further comprising indicia marked on the ball of compressed plant material.

7. The compressed plant material assembly of claim 1, wherein the compressed plant material assembly is disposed, with one or more additional plant material assemblies, such that two or more compressed plant material assemblies are disposed within a container comprising two or more separate compartments configured to hold the two or more compressed plant material assemblies in the two or more respective separate compartments.

8. An apparatus for forming a compressed plant material assembly, the apparatus comprising:
a base having an upper surface comprising a mold cavity, wherein the mold cavity comprises a major portion having a substantially dome-like shape, and wherein the mold cavity further comprises a minor portion having a substantially rectangular shape extending from the major portion along the upper surface;
a lid configured for removable placement on top of the base, wherein upon such placement the lid does not enclose the major portion of the mold cavity; and
a plunger comprising a proximal end and an opposite distal end, wherein the distal end comprises a plunger cavity having a substantially dome-like shape configured such that the plunger cavity and the major portion of the mold cavity together form a substantially spherical cavity.

9. The apparatus of claim 8, wherein the base further comprises a shelf surrounding at least a portion of the mold cavity, and wherein the lid rests against the shelf upon placement of the lid on top of the base.

10. The apparatus of claim 8, wherein the major portion of the mold cavity further comprises indentations in a form of indicia to be marked on the compressed plant material assembly.

11. The apparatus of claim 8, further comprising:
a wick comprising hemp; and
plant material comprising cannabis.

12. The apparatus of claim 8, wherein:
the base further comprises one or more additional mold cavities disposed in an array of two or more mold cavities, each of the one or more additional mold cavities comprising a major portion having a substantially dome-like shape, and each of the one or more additional mold cavities further comprises a minor portion having a substantially rectangular shape extending from the major portion along the upper surface.

13. The apparatus of claim 12 wherein:
upon removable placement of the lid, the lid encloses the minor portion of each of the two or more mold cavities, of the array of two or more mold cavities, and wherein upon such placement the lid does not enclose the major portion of each of the two or more mold cavities.

14. The apparatus of claim 8, wherein:
the base further comprises a chamber; and
the mold cavity is included in a mold configured to removably sit at least partially inside the chamber of the base.

15. The apparatus of claim 8, wherein the proximal end of the plunger comprises a scoop.

16. A method of forming a compressed plant material assembly, the method comprising:
providing a wick, plant material, a base, a lid, and a plunger,
wherein the base has an upper surface comprising a mold cavity, wherein the mold cavity comprises a major portion having a substantially dome-like shape, and wherein the mold cavity further comprises a minor portion having a substantially rectangular shape extending from the major portion along the upper surface,
wherein the plunger comprises a proximal end and an opposite distal end, and wherein the distal end comprises a plunger cavity having a substantially dome-like shape configured such that the plunger cavity and the major portion of the mold cavity together form a substantially spherical cavity;
laying the wick in the mold cavity, such that a portion of the wick is within the minor portion of the mold cavity, and such that a remainder of the wick is within the major portion of the mold cavity, such that the lid does not enclose the major portion of the mold cavity; and
compressing the plant material between the mold cavity and the plunger cavity using the plunger, thereby forming the compressed plant material assembly.

17. The method of claim 16, wherein the major portion of the mold cavity further comprises indentations in a form of indicia, and wherein the compressing further comprises marking the compressed plant material assembly with corresponding indicia.

18. The method of claim 16, wherein:
the wick comprises hemp; and
the plant material comprises cannabis.

19. The method of claim 16, wherein the method further comprises forming two or more compressed plant material assemblies by:
providing one or more additional wicks such that two or more wicks are provided;
providing one or more additional mold cavities such that two or more mold cavities are provided;
laying each of the two or more wicks in one of the two or more mold cavities, such that a portion of the wick is within the minor portion of the mold cavity, and such that a remainder of the wick is within the major portion of the mold cavity;
placing the lid on top of the base, such that the lid does not enclose the major portion of each of the two or more mold cavities; and
compressing the plant material between the two or more mold cavities and the plunger cavity using the plunger, thereby forming the two or more compressed plant material assemblies.

20. The method of claim 19, further comprising disposing the two or more compressed plant material assemblies within a container comprising two or more separate compartments configured to hold the two or more compressed plant material assemblies in the two or more respective separate compartments.
